(19) United States
(12) Patent Application Publication
(10) Pub. No.: US 2009/0264630 A1
(43) Pub. Date: Oct. 22, 2009

(54) METHOD OF SEPARATING MONOMERIC PROTEIN(S)

(75) Inventors: Kjell Eriksson, Uppsala (SE); Hans J. Johansson, Uppsala (SE); Urban Olsson, Gothenburg (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, Uppsala (SE)

(21) Appl. No.: 12/298,827

(22) PCT Filed: Jun. 5, 2007
(86) PCT No.: PCT/SE07/00542

§ 371 Date: Oct. 28, 2008

(30) Foreign Application Priority Data
Jun. 9, 2006 (SE) 0601297-5

(51) Int. Cl. C07K 1/16

(52) U.S. Cl. 530/413

(57) ABSTRACT

The present invention relates to a method of separating one or more monomeric proteins, such as monomeric antibodies, from a liquid. The method comprises providing a thiophilic aromatic chromatography matrix; contacting the liquid that comprises proteins with the matrix; and recovering at least one monomeric protein, such as a monomeric antibody, from the flow-through fraction. The proteins are advantageously monomeric antibodies.

### CROSS-REFERENCE TO RELATED APPLICATIONS

This application is a filing under 35 U.S.C. § 371 and claims priority to international patent application number PCT/SE2007/000542 filed Jun. 5, 2007, published on Dec. 13, 2007, as WO 2007/142578, which claims priority to patent application number 0601297-5 filed in Sweden on Jun. 9, 2006.

### FIELD OF THE INVENTION

The present invention relates to a method of separating proteins from liquids, such as in the purification of a monoclonal antibody from a cell culture liquid. The invention also encompasses a kit suitable for the separation of proteins, such as antibodies, from liquids.

### BACKGROUND OF THE INVENTION

The immune system is composed of many interdependent cell types that collectively protect the body from bacterial, parasitic, fungal, viral infections and from the growth of tumour cells. The guards of the immune system are macrophages that continually roam the bloodstream of their host. When challenged by infection or immunisation, macrophages respond by engulfing invaders marked with foreign molecules known as antigens. This event, mediated by helper T cells, sets forth a complicated chain of responses that result in the stimulation of B-cells. These B-cells, in turn, produce proteins called antibodies, which bind to the foreign invader. The binding event between antibody and antigen marks the foreign invader for destruction via phagocytosis or activation of the complement system. Five different classes of antibodies, or immunoglobulins, exist: IgA, IgD, IgE, IgG, and IgM. They differ not only in their physiological roles but also in their structures. From a structural point of view, IgG antibodies are a particular class of immunoglobulins that have been extensively studied, perhaps because of the dominant role they play in a mature immune response.

The biological activity, which the immunoglobulins possess, is today exploited in a range of different applications in the human and veterinary diagnostic, health care and therapeutic sector. In fact, in the last few years, monoclonal antibodies and recombinant antibody constructs have become the largest class of proteins currently investigated in clinical trials and receiving FDA approval as therapeutics and diagnostics. Complementary to expression systems and production strategies, purification protocols are designed to obtain highly pure antibodies in a simple and cost-efficient manner.

Traditional methods for isolation of immunoglobulins are based on selective reversible precipitation of the protein fraction comprising the immunoglobulins while leaving other groups of proteins in solution. Typical precipitation agents being ethanol, polyethylene glycol, lyotropic i.e. anti-chaotropic salts such as ammonium sulphate and potassium phosphate, and caprylic acid. Typically, these precipitation methods are giving very impure products while at the same time being time consuming and laborious. Furthermore, the addition of the precipitating agent to the raw material makes it difficult to use the supernatant for other purposes and creates a disposal problem, which is particularly relevant when speaking of large-scale purification of immunoglobulins.

Protein A and Protein G affinity chromatography are popular and widespread methods for isolation and purification of immunoglobulins, particularly for isolation of monoclonal antibodies, mainly due to the ease of use and the high purity obtained. Used as a capture step, followed by ion exchange, hydrophobic interaction, hydroxyapatite and/or gel filtration steps, especially protein A-based methods have become the antibody purification method of choice for many biopharmaceutical companies.

U.S. Pat. No. 5,429,746 (Smithkline Beecham Corp.) relates to the application of hydrophobic interaction chromatography combination chromatography to the purification of antibody molecule proteins. More specifically, a method for purifying monomeric IgG antibody from a mixture comprising said monomeric antibody and at least one of immunoglobulin aggregates, misfolded species, host cell protein or protein A is disclosed, which method comprises contacting said mixture with a hydrophobic interaction chromatographic support and selectively eluting the monomer from the support. Elution, whether stepwise or in the form of a gradient, can be accomplished in a variety of ways: (a) by changing the salt concentration, (b) by changing the polarity of the solvent or (c) by adding detergents. By decreasing salt concentration adsorbed proteins are eluted in order of increasing hydrophobicity. Changes in polarity may be affected by additions of solvents such as ethylene or propylene glycol or (iso)propanol, thereby decreasing the strength of the hydrophobic interactions. However, irrespective of the elution scheme used, it is well known in this field that adsorption followed by elution will inherently involve certain losses, i.e. a reduced yield.

U.S. Pat. No. 6,620,918 (Genentech Inc.) relates to a method for separating a polypeptide monomer, such as antibody monomers, from a mixture comprising dimers and/or multimers. More specifically, a method is disclosed, which consists essentially of applying a mixture to a cation-exchange or anion-exchange chromatography resin in a buffer, wherein if the resin is cation-exchange, the pH of the buffer is about 4-7, and wherein if the resin is anion-exchange, the pH of the buffer is about 6-9, and eluting the mixture at a gradient of about 0-1 M of an elution salt. The monomer is purified from the dimers or multimers or both present in the mixture, and the purified monomer is stated to have a purity of greater than 99.5% while the monomer yield is greater than 90%. A stated advantage of the '918 patent is that resins can be loaded to greater than 30 mg polypeptide/mL resin and still achieve excellent separations, and the separations are performed using either step or linear gradient elution. However, as stated above, binding the target compound in chromatography will inherently result in a reduced yield, as compared to having the target compound in the non-binding fraction.

WO 2006/024497 (Lonza) relates to affinity plus ion exchange chromatography for the purification of antibodies. More specifically, this patent application describes a method of purifying an antibody, preferably an IgG antibody, comprising the steps of purifying an antibody by means of protein A chromatography; loading the purified antibody comprising antibody aggregate and Protein A onto an ion exchange material under conditions which allow binding of the contaminating Protein A and resolution in the flow through of antibody aggregates from antibody monomer; and further fractionating the flow-through and harvesting at least one antibody monomer. The ion exchange material is preferably a quaternary amine-based anion exchanger, such as SEPHAROSE Q™ FF, which is a strong anion exchanger which is not susceptible to changes in pH of the loading/wash buffer.

Further, WO 2005/077130 (Tanox) relates to a method for the removal of aggregate proteins from recombinant samples using ion exchange chromatography. More specifically, in this patent application a process denoted a “bind-washout” process comprises choosing a resin suitable for manufacturing level purification of recombinant antibody; determining a pI value for the antibody monomer to be purified; determining a pH value and a salt concentration at which aggregates bind to the resin and wherein the antibody monomers interact weakly with the resin; and loading the recombinant antibody sample onto the chosen resin. Illustrative resins are all ion exchangers, such as Q SEPHAROSE™, DEAE SEPHAROSE™, SUPER-Q™ 650 and MACRO-PREP™ High Q. Like the Lonza patent application discussed above, here as well are Q groups used in the preferred embodiment.

Finally, Porath et al (J. Porath et al; FEBS Letters, vol. 185, p. 306, 1985) described how divinyl sulphone activated agarose coupled with various ligands comprising a free mercapto-group show specific binding of immunoglobulins in the presence of 0.5 M potassium sulphate. It was postulated that the sulphone group, from the vinyl sulphone spacer, and the resulting thioether in the ligand was a structural necessity to obtain the described specificity and capacity for binding of antibodies. Although the matrices described for such thiophilic aromatic chromatography generally show good performance, they also have a major disadvantage in that it is needed to add salts to the raw material to ensure efficient binding of the immunoglobulin.

U.S. Pat. No. 6,498,236 (Upfront Chromatography) relates to isolation of immunoglobulins. The method disclosed involves the steps of contacting a solution that comprises a negatively charged detergent and contains immunoglobulin(s) with a solid phase matrix, whereby at least a part of the immunoglobulins becomes bound to the solid phase matrix; and contacting the solid phase matrix with an eluent in order to liberate the immunoglobulin(s) from the solid phase matrix. The detergent present in the solution is believed to suppress the adherence of other biomolecules to the matrix, and is exemplified by octyl sulphate, bromphenol blue, octane sulphonate, sodium laurylsarcosinate, and hexane sulphonate.

However, there is still a need in this field of improved methods of separating antibodies, especially methods for separating monomeric antibodies from aggregates and the like without losses of yield.

### SUMMARY OF THE INVENTION

One aspect of the present invention is to provide a method of separating monomeric antibodies from a liquid, which method results in reduced losses, and consequently allows higher yields, than the prior art methods. This can be achieved by a method as defined by the appended claim 1, wherein the monomeric antibodies are maintained in the non-binding fraction while undesired components such as dimers, aggregates and misfolded species are adsorbed.

Another aspect of the invention is to provide a method of separating monomeric antibodies from a liquid, in which method the monomeric antibodies are not subjected to any salt addition and/or pH shift during the process. This can be achieved by a method as defined in the appended claims, wherein the monomeric antibodies are not bound to the chromatography matrix, and consequently do not require any elution.

A further aspect of the invention is to provide a kit for the separation of monomeric antibodies, such as monomeric monoclonal antibodies, from a liquid. A specific aspect is to provide such a kit for use in an aseptic pharmaceutical process. This can be achieved by a kit which comprises in separate compartments sterile components.

Other aspects and advantages will appear from the detailed description that follows.

### BRIEF DESCRIPTION OF THE DRAWINGS

FIG. 1 shows the chromatogram resulting from Example 1 below, wherein a monomeric monoclonal antibody is separated from dimers thereof in a liquid.

FIG. 2 shows the analytical size exclusion chromatography (SEC) of the Example 1 starting material, wherein the dimer concentration was 1.40% and the monomer concentration was 98.60.

FIG. 3 shows the analysis of the flow through pool from FIG. 1 (57 ml of flow-through, Fraction A2-B5), wherein the dimer concentration was less than 0.1%.

FIG. 4 shows the results of Example 2, wherein a MAb sample which has earlier been purified on MABSELECT™ SURE™ and CAPTO™ S is applied to thiophilic aromatic chromatography according to the invention.

### DEFINITIONS

The terms “antibody” and “immunoglobulin” are used herein interchangeably. It is understood that in the present context, the term “antibody” includes complete antibodies as well as fragments thereof, such as Fab fragments, and fusion proteins, such as Fc fusion proteins, comprising all or part of an antibody.

The term “aggregate” means a non-covalent association of identical protein entities. The term “chromatography matrix” is used herein for an insoluble carrier to which ligands have been attached.

The term “ligand” means herein molecules or compounds capable of interaction with other compounds, such as antibodies and/or contaminants.

The term “thiophilic” aromatic chromatography means chromatography using a matrix wherein the ligands comprise at least one sulphuric functionality and at least one aromatic functionality.

The term “aromatic” group refers to a group, wherein the number of π electrons can be calculated according to Huckels rule: (4n+2), wherein n is a positive integer or zero. The term “spacer arm” means herein an element that distances a ligand from the support of a separation matrix.

The term “surface” when used in the context of a porous support embraces the pore surfaces as well as to the actual outer surfaces.

The term “mobile phase” is used herein interchangeably with “adsorption buffer”.

The term “eluent” is used in its conventional meaning in this field, i.e. a buffer of suitable pH and/or ionic strength to release one or more compounds from a separation matrix.

The term “gradient elution” means gradually changing the conditions from binding to non-binding conditions.

The term “flow-through” as used herein refers to the fraction of the mobile phase that does not bind to the chromatography matrix, and consequently does not require any elution. The “flow-through” is sometimes denoted the “non-binding” fraction.

### DETAILED DESCRIPTION OF THE INVENTION

Thus, in a first aspect, the present invention relates to a method of separating one or more monomeric proteins from a liquid, which method comprises providing a thiophilic aromatic chromatography matrix; contacting the liquid that comprises proteins with the matrix; and recovering the protein(s) from the flow-through fraction. The protein may be any protein, and is more particularly a protein known to form aggregates. In an advantageous embodiment, the protein is albumin.

In an advantageous embodiment, the invention relates to a method of separating one or more monomeric antibodies from a liquid, which method comprises providing a thiophilic aromatic chromatography matrix; contacting the liquid that comprises antibodies with the matrix; and recovering the monomeric antibodies from the flow-through fraction. In an advantageous embodiment, the separated monomeric antibody is monoclonal. In the present context, it is understood that “monomeric” refers to one single antibody, which as is well known is comprised of four polypeptide chains (2 heavy chains and 2 light chains). Such a monomeric IgG is commonly of a size in the region of 150,000 Daltons.

The method according to the invention may be operated as liquid chromatography, wherein the mobile phase i.e. the liquid from which monomeric proteins, such as the monomeric antibodies, are separated is passed across a chromatography column comprising matrix; or as a batch process, wherein the mobile phase is added to chromatography matrix in a vessel. In either case, the term “flow through” fraction is used herein for the liquid removed from the chromatography matrix, which liquid comprises the non-adsorbed material.

As appears from the above, the present method comprises contacting a mobile phase, which comprises the liquid from which monomeric proteins, such as monomeric antibodies, are to be separated and optionally a buffer, with a thiophilic aromatic chromatography matrix the thiophilic aromatic chromatography matrix comprises at least one sulphuric functionality and at least one aromatic functionality. The thiophilic aromatic ligands used in this embodiment may be described by the general formula -X-S—(CH₂)ₙ—R, wherein X is a spacer arm coupled to the carrier; n is an integer 0-10, such as 0, 1 or 2; and R is an aromatic group.

In a first embodiment, the aromatic group R is substituted, and in an alternative embodiment, it is non-substituted. In an advantageous embodiment, R comprises at least one nitrogen atom in its ring structure. Such systems are e.g. as described e.g. in U.S. Pat. No. 5,942,463 (Oscarsson et al). Thus, R may be a substituted or non-substituted aromatic carbon chain of 1-10 atoms, such as 2-6. A commercially available thiophilic aromatic chromatography matrix is PLASMIDSELECT™ (GE Healthcare, Uppsala, Sweden). Thus, illustrative ligands are e.g. 2-mercaptopyridine, 2-mercaptopyrimidine, and 2-mercaptothiazoline.

According to the present invention, undesired species such as dimeric proteins; aggregates formed by proteins binding to proteins; and host cell proteins may be adsorbed to the matrix. In an advantageous embodiment, when the monomeric protein is a monomeric antibody, undesired species such as dimeric antibodies; aggregates formed by antibodies binding to antibodies, or antibodies binding to other protein; and host cell proteins are adsorbed to the matrix. In a specific embodiment, which will be discussed in more detail below, when the method of the invention is used subsequent to a preceding chromatography step, ligands that have leaked from that preceding step are also adsorbed to the matrix. For example, if the preceding step utilizes affinity chromatography, such as Protein A ligands, leaked Protein A is adsorbed in the present method. In addition, other contaminating species such as nutrients added during fermentation, endotoxins etc are also removed by adsorption to the chromatography matrix.

The liquid from which the monomeric proteins, such as the monomeric antibodies, are separated may be any biological liquid, such as serum of immunized animals, ascites fluid, hybridoma or myeloma supernatants, conditioned media derived from culturing a recombinant cell line that expresses the proteins such as an immunoglobulin molecule and from all cell extracts of immunoglobulin producing cells. Thus, in one embodiment, the liquid comprise a cell culture liquid. If required, the liquid may be combined with a suitable buffer.

Further, the liquid from which the monomeric proteins, such as the monomeric antibodies, are separated may comprise an eluent from a preceding affinity chromatography step. Thus, the method according to the invention may be used as a second, third or further process step, advantageously as the step commonly denoted “polishing”, wherein the last, minor amounts of undesired impurities are finally removed from the preparation. In one embodiment, one step preceding the present method utilises Protein A ligands, or ligands that comprise parts or all of Protein A, or a genetically manipulated form of Protein A or a part thereof. Chromatography matrices comprising Protein A ligands are well known in the art and frequently used for capture of antibodies. Illustrative examples are MABSELECT™ (GE Healthcare, Uppsala, Sweden) and PROSEP A™ (Millipore).

In a second aspect, the invention relates to the use of a thiophilic aromatic chromatography matrix to separate one or more monomeric proteins, such as monomeric antibodies, from other components of a liquid, which use comprises adsorbing said other components to a thiophilic aromatic chromatography matrix. The details above in the context of the method as such may apply to the present use as well.

In a third aspect, the invention relates to a kit for the separation of one or more monomeric proteins from undesired species in a liquid, which kit comprises, in separate compartments, a chromatography column packed with a thiophilic aromatic chromatography matrix; one or more buffers; and written instructions for adsorbing certain components to the matrix. In an advantageous embodiment, at least one monomeric protein is an antibody. The details above in the context of the method may apply to this aspect as well. In an advantageous embodiment, the kit, or at least the chromatography matrix, has been sterilized. Such a kit is advantageously used in the pharmaceutical or diagnostic industry, where purity is of utmost importance.

As the skilled person will appreciate, a chromatography matrix which has been used according to the present invention may be regenerated by elution of the undesired species by common elution.

### DETAILED DESCRIPTION OF THE DRAWINGS

FIG. 1 shows the chromatogram resulting from Example 1 below, wherein a monomeric monoclonal antibody is separated from dimers thereof in a liquid.

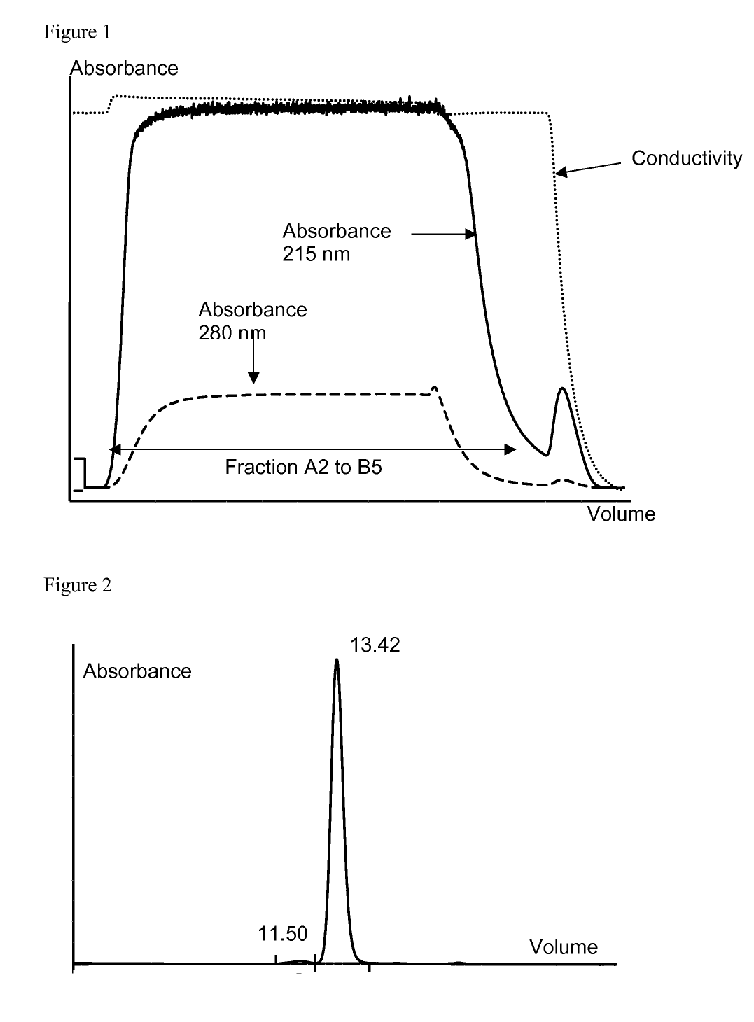

FIG. 2 shows the analytical size exclusion chromatography (SEC) of the Example 1 start material, wherein the dimer concentration was 1.40% (Peak 1 in FIG. 2 [elution volume 11.50 ml]) and the monomer concentration was 98.60% (Peak 2 in FIG. 2 [elution volume 13.42 ml]).

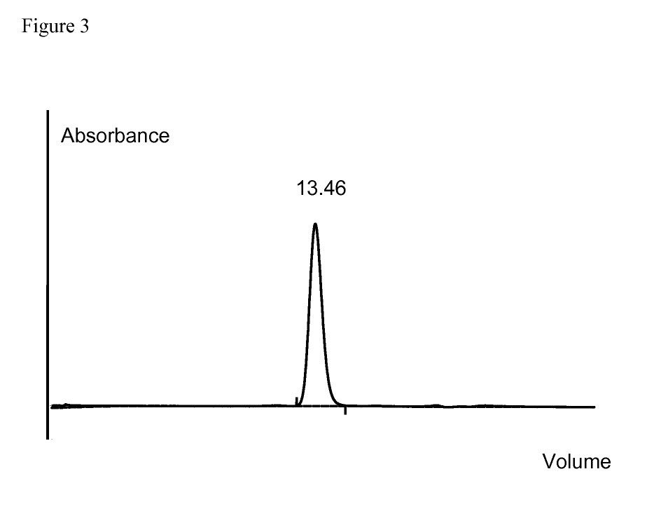

FIG. 3 shows the analysis of the flow through pool from FIG. 1 (57 ml of flow-through, Fraction A2-B5), and indicates less than 0.1% dimeric MAb, i.e. very close to 0% dimers therein. Thus, as expected, the monomeric antibody passed through the column without binding, while the dimeric antibody was adsorbed to the column, i.e. a reduction of amount of dimers from 1.40% to less than 0.1% in the product pool (fractions A2 to B5 in FIG. 1). As deduced from FIG. 3, at retention (ml) 13.46, monomeric Mab, corresponds to approximately 100%.

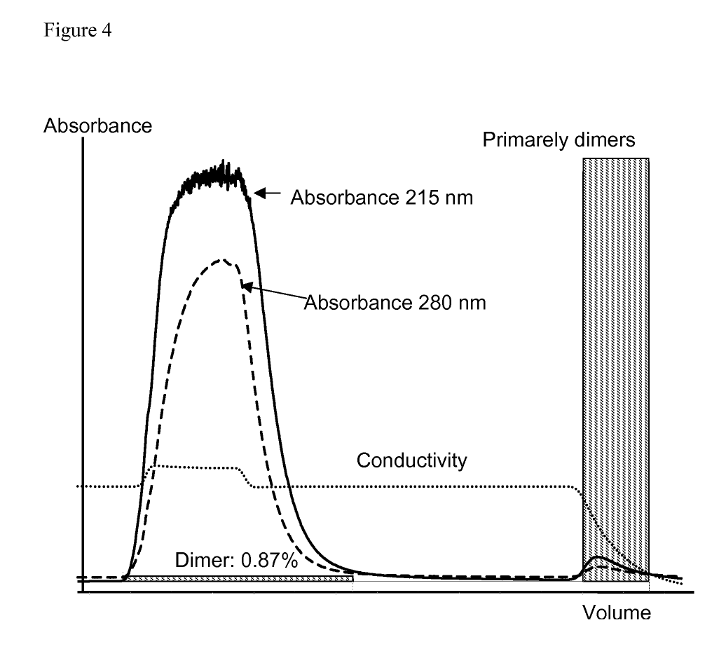

FIG. 4 shows the results of Example 2, wherein a MAb sample which has earlier been purified on MABSELECT™ SURE™ and CAPTO™ S is applied to thiophilic aromatic chromatography on PLASMIDSELECT™. Even though the conditions were not optimised, the amount of dimer/aggregates was reduced to a level below 1%, from a start material containing 2.5% dimer plus aggregate.

### EXAMPLES

Below the present invention will be disclosed by way of examples, which are intended solely for illustrative purposes and should not be construed as limiting the present invention as defined in the appended claims. All references mentioned below or elsewhere in the present application are hereby included by reference.

### Example 1

### Purification of Monoclonal Antibody Monomers from Dimers

This example illustrates how monomers of monoclonal antibody can be purified from dimers thereof according to the invention, in non-binding (flow-through) mode using a thiophilic aromatic chromatography matrix. The results of the separation are shown in FIG. 1.

Column: TRICORN™ 5/100 (GE Healthcare, Uppsala, Sweden) (volume 2 ml)
Chromatography matrix: PLASMIDSELECT™ (GE Healthcare, Uppsala, Sweden)
Flow: 0.6 ml/min
Fraction volume: 3.0 ml

### Det: UV 215 nm and 280

Buffers: Equilibration and in sample—330 mM potassium phosphate, pH 6.8 (conductivity of 39 mS/cm). The elution of dimers/other aggregates was done with a step elution with water

The liquid applied to the herein used thiophilic aromatic chromatography matrix (PLASMIDSELECT™, GE Healthcare, Uppsala, Sweden) originates from an anion exchanger (CAPTO Q™, GE Healthcare, Uppsala, Sweden), to which an eluate from an affinity chromatography matrix (MABSELECT™ SURE™, GE Healthcare, Uppsala, Sweden) had been applied.

46 ml of as mAb (IgG) solution, total amount: 122.8 mg.
Pool: Fractions (A2 to B5)-57 ml, amount monomeric IgG: 114 mg
Yield 92.8%, determined spectrophotometrically by measurements at 280 nm.
% Dimers in Start material: 1.40%
% Dimers in Pool: <0.1% (or not detected)
Dimer concentration determined with Size Exclusion Chromatography (SEC), using a Superdex 200 column.

FIGS. 2 and 3 are analytical chromatograms

### Example 2

### Thiophilic Aromatic Chromatography Following Affinity Chromatography and Ion Exchange

This example illustrates how the present invention can be used as a third step for polishing of monoclonal antibodies in a purification process. The liquid applied to the herein used thiophilic aromatic chromatography matrix (PLASMIDSELECT™, GE Healthcare, Uppsala, Sweden) originates from a cation exchanger (CAPTO S™, GE Healthcare, Uppsala, Sweden), to which an eluate from an affinity chromatography matrix (MABSELECT™ SURE™, GE Healthcare, Uppsala, Sweden) had been applied. The results are presented in FIG. 4.

In this example thiophilic aromatic adsorption chromatography remove dimers and other larger aggregates from a monoclonal antibody solution. This is done by the use of PLASMIDSELECT™ (GE Healthcare, Uppsala, Sweden), in flow-through mode. Using a potassium phosphate buffer at neutral pH with a moderate concentration (0.1 to 0.4 M), the dimers and aggregates bind more strongly than the monomers and can thus be separated from these monomers.

Column: TRICORN™ (GE Healthcare, Uppsala, Sweden) 5/100 (approx 2 ml)
Chromatography matrix: PLASMIDSELECT™ (GE Healthcare, Uppsala, Sweden)
Flow 0.6 ml/min.

The MAb sample was loaded in 0.4 M potassium phosphate, pH 6.8. The percentage of dimer in respective pool is indicated in FIG. 4. Fraction A2-A8 contains 0.87% dimer.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A method of separating one or more monomeric proteins from a liquid, which method comprises:
providing a thiophilic aromatic chromatography matrix;
contacting the liquid that comprises said protein(s) with the matrix; and
recovering the monomeric protein(s) from the flow-through fraction.

2. The method of claim 1, wherein at least one monomeric protein is a monomeric antibody.

3. The method of claim 2, wherein the antibody is a monoclonal antibody.

4. The method of claim 1, wherein the thiophilic aromatic chromatography matrix comprises at least one sulphuric functionality and at least one aromatic functionality.

5. The method of claim 1, wherein undesired species such as dimeric proteins; aggregates formed by proteins binding to proteins; and host cell proteins are adsorbed to the matrix.

6. The method of claim 1, wherein the liquid is a cell culture liquid.

7. The method of claim 1, wherein the liquid comprises an eluent from a preceding affinity chromatography step.

8. The method of claim 7, wherein the preceding affinity step utilises Protein A ligands.

9-10. (canceled)

11. A kit for the separation of one or more monomeric proteins from undesired species of a liquid, which kit comprises, in separate compartments, a chromatography column packed with a thiophilic aromatic chromatography matrix; one or more buffers; and written instructions for adsorbing undesired components to the matrix.

12. The kit of claim 11, wherein at least one monomeric protein is an antibody, such as a monoclonal antibody.

13. The kit of claim 11, wherein the chromatography matrix has been sterilized.
